(19) United States
(12) Patent Application Publication
(10) Pub. No.: US 2010/0119465 A1
(43) Pub. Date: May 13, 2010

(54) Enhanced photoactivity of semiconductors

(75) Inventors: Laura A. Spaulding, Allendale, NJ (US); A. Christopher Pattillo, Allendale, NJ (US); Patricia L. Scott, Allendale, NJ (US)

Correspondence Address: Charles, N.J. Ruggiero, 10th Floor, One Landmark Square, Stamford, CT, 06901-2682, US

(73) Assignee: Playtex Products, LLC

(21) Appl. No.: 12/586,101

(22) Filed: Sep. 17, 2009

Related U.S. Application Data
(63) Continuation-in-part of application No. 12/483,943, filed on Jun. 12, 2009.
(60) Provisional application No. 61/131,982, filed on Jun. 13, 2008.

(51) Int. Cl. A61K 8/29; A61K 8/27; A61Q 17/04; A61K 8/30

(52) U.S. Cl. 424/60; 424/59

(57) ABSTRACT

The present disclosure provides a way to enhance the photoactivity of a semiconductor. By dispersing the semiconductor with a compound having multiple phenyl rings in a polar carrier oil, the semiconductor exhibits greatly improved photoactivity and/or SPF over dispersions comprising the semiconductor alone.

### CROSS-REFERENCE TO RELATED APPLICATIONS

The present application is a continuation-in-part of U.S. patent application Ser. No. 12/483,943, filed on Jun. 12, 2009, which in turn claims the benefit of U.S. Provisional Application No. 61/131,982, filed on Jun. 13, 2008.

### BACKGROUND OF THE DISCLOSURE

1. Field of the Disclosure

The present disclosure relates to enhancing the photoactivity of semiconductors, such as the sun protection factor (SPF), and/or the UVA absorption. In particular, the present disclosure relates to enhancing these properties of semiconductors by dispersing them with chemical compounds having multiple phenyl rings, in a polar organic carrier oil.

2. Description of the Related Art

Photoactivity of a semiconductor refers to the material's ability to absorb photons of light energy. Enhancing the photoactivity of the semiconductor can thus improve its performance in a variety of electronic applications. Some semiconductors are also useful as sunscreen active agents. It is always a goal in the field of suncare to either use less sunscreen active material while maintaining a desired level of SPF and/or UVA absorption, or to achieve a very high SPF or UVA absorption rate overall. Thus, there is a need to a way of enhancing the photoactivity of semiconductor materials, which can boost the SPF, and/or UVA absorption of these materials.

### SUMMARY OF THE DISCLOSURE

The present disclosure thus provides dispersions and compositions that enhance the photoactivity—i.e., the SPF and/or UVA absorption—of semiconductors.

In one embodiment, the present disclosure provides a dispersion comprising a semiconductor, a compound having multiple phenyl rings, and a polar carrier oil.

In another embodiment, the present disclosure provides a sunscreen composition, comprising a sunscreen and a dispersion. The dispersion comprises a semiconductor, a compound having multiple phenyl rings, and a polar carrier oil.

### BRIEF DESCRIPTION OF THE DRAWINGS

FIG. 1 is a conceptual drawing of a dispersion of the present disclosure;

FIGS. 2-4 show UV absorption data for several dispersions of the present disclosure;

FIGS. 5-7 show the UV absorption data of FIGS. 2-4, respectively, focused on the UVA range; and

FIGS. 8-11 show graphs illustrating aggregate particle sizes of the semiconductors when in some of the dispersions of the present disclosure.

### DETAILED DESCRIPTION OF THE DISCLOSURE

The present disclosure has unexpectedly discovered that when one or more semiconductors, such as zinc oxide or titanium dioxide, are combined with one or more compounds containing multiple (i.e., at least two) phenyl rings, the photoactivity of the semiconductors are significantly enhanced. This is contrary to the common understanding of how to boost these characteristics. Previously, it was thought that to enhance these properties, dopants would have to be added to the crystal lattice structure of the semiconductor. The present disclosure, by contrast, has discovered that the photoactivity can be increased by combining the semiconductor, with the aforementioned compounds having multiple phenyl rings (hereinafter “phenyl compounds”), in a dispersion. Thus, these properties of a semiconductor can be enhanced without undergoing complicated and costly doping processes. This results in less semiconductor material being required for a particular application, or in dispersions and compositions having high SPF or UVA absorption values that were previously not thought possible.

The present disclosure thus provides a dispersion comprising a semiconductor, a phenyl compound, and a polar carrier oil as a solvent. In one embodiment, the semiconductor can be one or more semiconductors selected from the group recited in the Wikipedia page, “List of semiconductor materials,” found at http://en.wikipedia.org/wiki/List_of_semiconductor_materials, which is herein incorporated by reference. In another embodiment, the semiconductor is selected from the group consisting of titanium dioxide, zinc oxide, (both of which can double as sunscreen actives) and a combination thereof. The semiconductor can be present in an amount of about 0.50 wt % to about 50 wt %, or about 20 wt % to about 30 wt %, based on the total weight of the dispersion. The semiconductor can also be present in precisely those amounts, i.e. 0.50 wt % to 50 wt %, or 20 wt % to 30 wt %, based on the total weight of the dispersion. When present, the zinc oxide used in the present disclosure can be acquired from a number of vendors, such as the Umicore Group or BASF.

In one embodiment, the one or more phenyl compounds can be selected from the group consisting of benzene sulfonic acids, or salts thereof, styrenic block copolymers with a hydrogenated midblock of styrene-ethylene/butylene-styrene (SEBS), styrenic block copolymers with a hydrogenated midblock of styrene-ethylene/propylene-styrene (SEPS), styrene/butadiene/styrene (SBS) block copolymers, styrene/isoprene/styrene (SIS) block copolymers, an ethylene/butadiene/styrene (EBS) block copolymer, an ethylene/propylene/styrene (EPS) block copolymer, and any derivatives or combinations thereof. Examples of the SEBS, SEPS, SBS, SIS, EBS, or EPS block copolymers are the Kraton® D and Kraton® G series from Kraton Polymers, for example Kraton® D1650. An example of a benzene sulfonic acid salt is sodium polystyrene benzene sulfonate (available, for example, as Flexan® II, from AzkoNobel).

The phenyl compound can be present in an amount of about 0.05 wt % to about 10 wt %, or about 0.10 wt % to about 5 wt %, based on the total weight of the dispersion. The phenyl compound can also be present in precisely those amounts, i.e. 0.05 wt % to 10 wt %, or 0.10 wt % to 5 wt %, based on the total weight of the dispersion.

The polar carrier oil can be one or more oils suitable for the purpose of allowing the phenyl compounds to interact with the semiconductors in the manner discussed below. In one embodiment, the polar carrier oil can be one or more esters. The esters can be benzoate or non-benzoate esters, with alkyl chain lengths that are branched or non-branched. In another embodiment, the polar carrier oil can be selected from the group consisting of isopropyl myristate, butyloctyl salicylate, octisalate, isononyl isonanoate, and ethylhexyl benzoate, or any combinations thereof. Examples of commercially available esters suitable for use in the dispersion of the present disclosure include, but are not limited to, the Finsolv® benzoate esters available from Innospec Active Chemicals, the Schercemol® or Hydramol® esters available from the Lubrizol Corporation, or the Crodamol® esters available from Croda Worldwide.

The polar carrier oil can be present in an amount of about 40 wt % to about 99.50 wt %, or about 75 wt % to about 95 wt %, based on the total weight of the dispersion. The polar carrier oil can also be present in precisely those amounts, i.e. 65 wt % to 99.50 wt %, or 75 wt % to 95 wt %, based on the total weight of the dispersion.

Without being bound by a specific theory, it is believed that the phenyl compound causes a large electronegative cloud to come into contact with the surface of the semiconductor, thus causing an increase in the photoactivity of the semiconductor. Traditionally, semiconductors are often doped in an attempt to help the electrons residing in the valence bands of the semiconductor material cross the band gap to the conduction bands when the semiconductor is exposed to light, which enhances the photoactivity of the semiconductor. The dopant donates valence electrons, thus making the migration across the band gap easier.

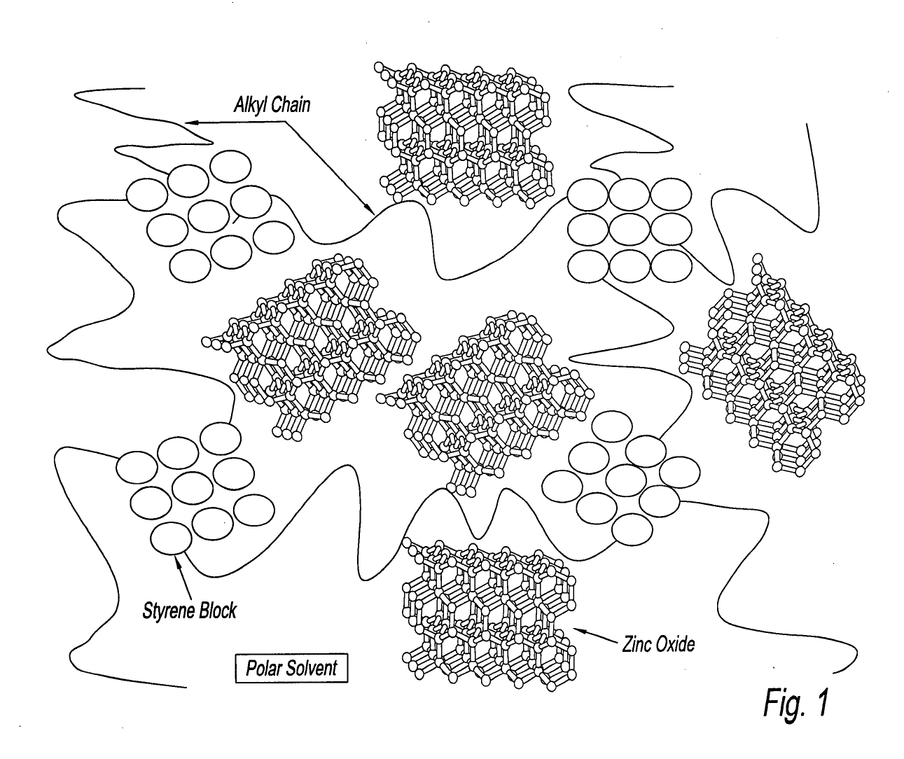

In the dispersions of the present disclosure, however, there is no need to dope the semiconductor with other metallic cations. The phenyl compounds act as external dopants to the semiconductor, as opposed to traditional dopants (sometimes referred to as “extrinsic” dopants), which would be located within the semiconductor crystal lattice. The large electronegative cloud provided by the phenyl compound may facilitate the “jump” of valence electrons to the conduction bands, in part because in some semiconductors, there are no electron orbitals in the band gap region. Also, this relatively large, strong electronegative cloud has the ability to polarize the filled d-orbitals of some semiconductors, thus possibly distorting the orbitals and ultimately affecting the band gap region, thereby facilitating electron flow. It is possible that the phenyl compounds may be acting as n-type dopants (donating electrons) to enhance the n-type characteristics of the semiconductor. A conceptual drawing of this concept is shown in FIG. 1.

There are many useful applications for the dispersions of the present disclosure, due to their enhanced performance characteristics. For example, such a dispersion would be extremely valuable in any number of electronics applications, such as in computing devices, cellular phones, batteries, optoelectronic devices, photovoltaic cells, and others. The enhanced SPF and UVA absorption of this dispersion is particularly valuable in the field of personal care and sunscreen compositions.

To observe the effects that the dispersions of the present disclosure can have on the SPF of a semiconductor, several different dispersions were tested, as shown in Table 1 below. The dispersions were all applied at a coverage rate of 1.16 mg/cm². In the third and fourth samples listed below, zinc oxide was applied a rate of 0.2332 mg/cm², and the IPM and 10% Kraton 1650 in IPM each being applied at a rate of 0.9268 mg/cm². The samples were then analyzed with a Labsphere 1000S UV Transmittance Analyzer for SPF value.

TABLE 1

| Composition | SPF |
|---|---|
| Isopropyl Myristate (IPM)(Control) | 0.73 |
| 10% Kraton 1650 in IPM | 0.73 |
| Zinc Oxide (ZnO) + IPM | 8.63 |
| ZnO + 10% Kraton 1650 in IPM | 28.95 |

Thus, as shown above, the Kraton 1650 polymer greatly enhances the SPF of zinc oxide, when in a dispersion with IPM. This is a very unexpected result. It was not thought possible to enhance the SPF of a semiconductor, such as zinc oxide, with the use of a phenyl compound, such as Kraton 1560, because as shown above, the latter has no SPF value on its own. Yet, when the Kraton 1560 is added to the zinc oxide, the resulting dispersion has a greatly enhanced SPF value when compared to a dispersion having zinc oxide alone. The dispersions discussed in the present disclosure can thus be used in sunscreen formulations, where they will provide significantly enhanced SPF characteristics. The resultant sunscreen formulations can have high SPF and UVA/UVB absorption values, while only requiring smaller amounts of the semiconductor materials in the formulation. Alternatively, the dispersions of the present disclosure can be used to create compositions having high SPF and UVA absorption values that were not previously thought possible.

The dispersions of the present disclosure can be used alone, or combined into a sunscreen composition with one or more additional sunscreen actives, other than the semiconductors that also function as sunscreen actives. The one or more additional sunscreen actives can be, but are not limited to, cinnamates, octisalate, p-aminobenzoic acid, its salts and its derivatives (ethyl, isobutyl, glyceryl esters; p-dimethylaminobenzoic acid); anthranilates (o-aminobenzoates; methyl, menthyl, phenyl, benzyl, phenylethyl, linalyl, terpinyl, and cyclohexenyl esters), salicylates (octyl, amyl, phenyl, benzyl, menthyl (homosalate), glyceryl, and dipropyleneglycol esters), cinnamic acid derivatives (menthyl and benzyl esters, alpha-phenyl cinnamonitrile; butyl cinnamoyl pyruvate), dihydroxycinnamic acid derivatives (umbelliferone, methylumbelliferone, methylaceto-umbelliferone), camphor derivatives (3-benzylidene, 4-methylbenzylidene, polyacrylamidomethyl benzylidene, benzalkonium methosulfate, benzylidene camphor sulfonic acid, and terephthalylidene dicamphor sulfonic acid), trihydroxycinnamic acid derivatives (esculetin, methylesculetin, daphnetin, and the glucosides, esculin and daphnin), hydrocarbons (diphenylbutadiene, stilbene), dibenzalacetone and benzalacetophenone, naptholsulfonates (sodium salts of 2-naphthol-3,6-disulfonic and of 2-naphthol-6,8-disulfonic acids), dihydroxy-naphthoic acid and its salts, o- and p-hydroxydiphenyldisulfonates, coumarin derivatives (7-hydroxy, 7-methyl, 3-phenyl), diazoles (2-acetyl-3-bromoindazole, phenyl benzoxazole, methyl naphthoxazole, various aryl benzothiazoles), quinine salts (bisulfate, sulfate, chloride, oleate, and tannate), quinoline derivatives (8-hydroxyquinoline salts, 2-phenylquinoline), hydroxy- or methoxy-substituted benzophenones, uric and vilouric acids, tannic acid and its derivatives, hydroquinone, benzophenones (oxybenzone, sulisobenzone, dioxybenzone, benzoresorcinol, 2,2′,4,4′-tetrahydroxybenzophenone, 2,2′-dihydroxy-4,4′-dimethoxybenzophenone, octabenzone), dibenzoylmethane derivatives, avobenzone, 4-isopropyldibenzoylmethane, butylmethoxydibenzoylmethane, 4-isopropyl-dibenzoylmethane, octocrylene, drometrizole trisiloxane, bemotrizinol (sold under the trade name Tinasorb®), ecamsule (sold under the trade name Mexoryl®), and any combinations thereof.

The one or more additional sunscreen actives can be present in an amount of about 1 wt % to about 35 wt %, or about 3 wt % to about 12 wt % of the composition, or in precisely these amounts, i.e. 1 wt % to 35 wt %, or 3 wt % to 12 wt % of the composition. The composition may also comprise one or more additives, such as emulsifiers, thickeners, emollients, pH adjusters, stabilizers, and film formers. The dispersion can be present in the sunscreen composition so that the amount of semiconductor present in the sunscreen composition is between about 1 wt % and about 20 wt %, or about 1 wt % and about 5 wt %, or in precisely these amounts, i.e. between 1 wt % and 20 wt %, or 1 wt % and 5 wt %.

The dispersion of the present disclosure can be in an oil-in-water, or water-in-oil form. Any final products using the compositions or dispersions of the present disclosure can take the form of sprays, sticks, gels, lotions, creams, or any other form for suitable delivery.

The following data further illustrates the advantages of the dispersions of the present disclosure.

### Materials Used

### ZnO Powder 45 nm and 75 nm Particle Size

Ethylhexyl salicylate (OS), butyloctyl salicylate (BHB), ethylhexyl benzoate (EB),
isopropyl myristate (IPM), and isononyl isonanoate (II)

### Dispersions:

### 5% Kraton G1650 (K, Kraton) in Isopropyl Myristate (IPM)

### 5% Kraton G1650 (K, Kraton) in Isononyl Isonanoate (II)

### 5% Kraton G1650 (K, Kraton) in Ethylhexyl Benzoate (EB)

### 5% Kraton G1650 (K, Kraton) in Butyloctyl Salicylate (BHB)

### 5% Kraton G1650 (K, Kraton) in Ethylhexyl Salicylate (OS)

### ZnO (IPM) 20% ZnO in Isopropyl Myristate

### ZnO (II) 20% ZnO in Isononyl Isonanoate

### ZnO (EB) 20% ZnO in Ethylhexyl Benzoate

### ZnO (K EB) 20% ZnO in 5% Kraton G1650/Ethylhexyl Benzoate Blend

### ZnO (BHB) 20% ZnO in Butyloctyl Salicylate

### ZnO (K BHB) 20% ZnO in 5% Kraton G1650/Butyloctyl Salicylate Blend

### ZnO (OS) 20% ZnO in Octisalate (Ethylhexyl Salicylate)

### ZnO (K OS) 20% ZnO in 5% Kraton G1650/Octisalate Blend

For the ZnO dispersions, a Ross homogenizer was utilized to break-up the ZnO agglomerates to help maximize content uniformity within and among the sample dispersions.

Results from SPF Studies

The in-vitro SPF results summarized in Table II demonstrate the effect of solvent polarity on ZnO, and surprisingly, the substantial boost in SPF from the combination of the solvent and Kraton polymer.

TABLE II
Effect of ZnO (45 nm)/Kraton/Solvent on In-Vitro SPF

SPF (units)

| Sample | IPM | II | EB | BHB | OS |
|---|---|---|---|---|---|
| Neat | 0.99 | 0.99 | 1.04 | 15.05 | 16.03 |
| +Kraton | 1.02 | 0.97 | 1.00 | 14.82 | 15.37 |
| +ZnO | 6.84 | 14.37 | 21.58 | 78.88 | 72.80 |
| +Kraton + ZnO | 62.46 | 55.11 | 66.16 | 123.48 | 133.04 |

Kraton polymer is a solid material that must be dispersed in solvent. The data in Table II suggests that the Kraton polymer not only has no SPF value on its own, but may actually slightly depress the SPF of the polar solvent. The dielectric constant for the Kraton/BHB and Kraton/OS blends without the presence of ZnO are 5.21 and 5.98, respectively, as shown in Table V, discussed below. Therefore, the dielectric constant data supports the slightly lower SPF trend noted for adding the Kraton polymer to the polar solvent.

Although ZnO in the Kraton/solvent blends does increase the SPF, it is the significance of the increase produced by the combination of the ZnO lattice structure in close proximity to the Kraton web-like matrix that becomes important. For the combination of ZnO/Kraton/BHB and ZnO/Kraton/OS dispersions, there was a remarkable 8.33 and 8.66 fold increase in SPF response versus the corresponding Kraton/solvent blend. Additionally, the combination of ZnO/Kraton versus ZnO alone showed a remarkable SPF unit increase by 56.5% in BHB, and 82.7% increase in OS solvents.

The data presented in Table III below demonstrates that the increase in observed SPF for the ZnO/Kraton/polar solvent dispersions were unexpectedly synergistic and not just additive. The Theoretical SPF is the sum of the SPF values for the dispersions for the Kraton and ZnO individually. So, for example, with the IPM dispersion, the Theoretical SPF would be 1.02+6.84=7.86, based on the values from Table III above. However, SPF values increased 30-55 units above what would normally be expected from an additive effect. The synergistic SPF effect was +31.8% for ZnO/Kraton in BHB, and +50.9% for ZnO/Kraton in OS. Hence, the photoactivity of ZnO is synergistically enhanced by the combination of Kraton and polar solvent.

TABLE III
Determination of Additive versus Synergistic
Effect for In-Vitro SPF with ZnO

| | SPF (units) | | | | |
|---|---|---|---|---|---|
| Sample | IPM | II | EB | BHB | OS |
| Theoretical SPF | 7.86 | 15.34 | 22.58 | 93.70 | 88.17 |
| Solvent + Kraton + ZnO | | | | | |
| Observed SPF | 62.46 | 55.11 | 66.16 | 123.48 | 133.04 |
| Solvent + Kraton + ZnO | | | | | |
| SPF Unit Difference | +54.60 | +39.77 | +43.31 | +29.78 | +44.87 |

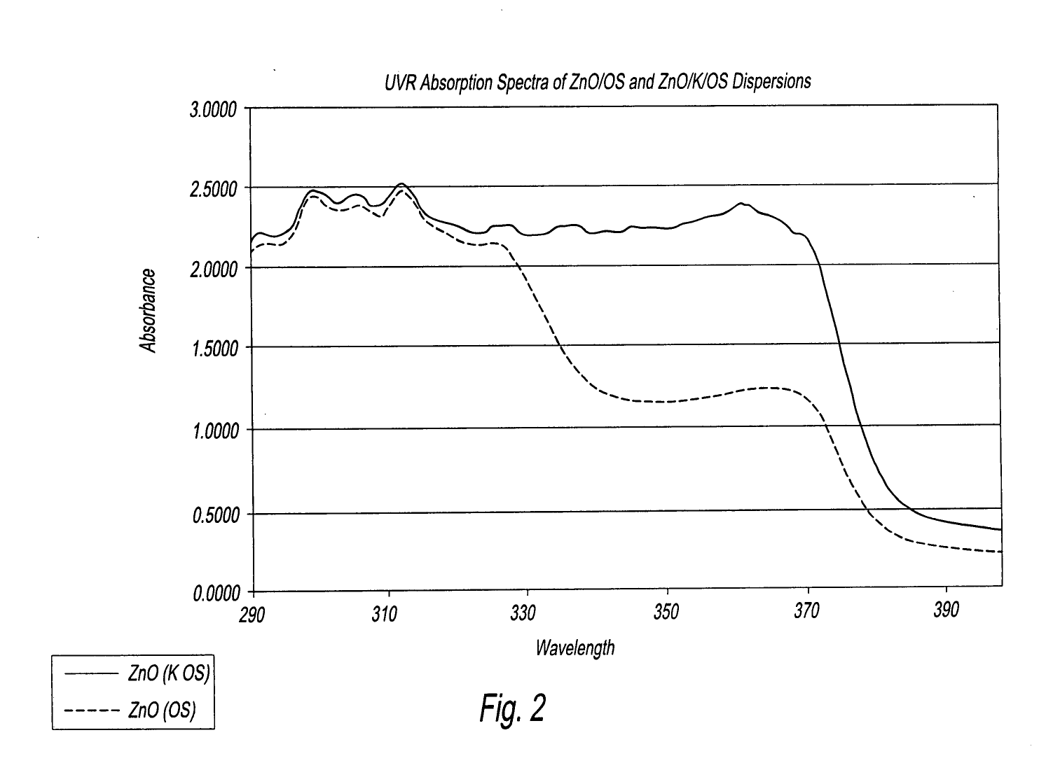

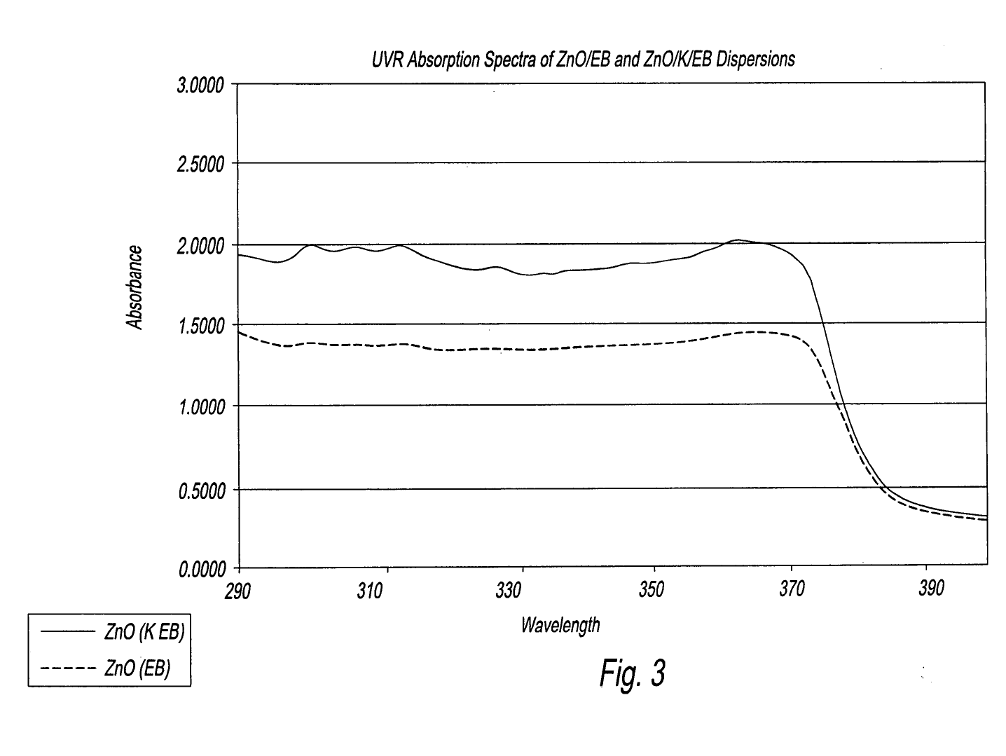

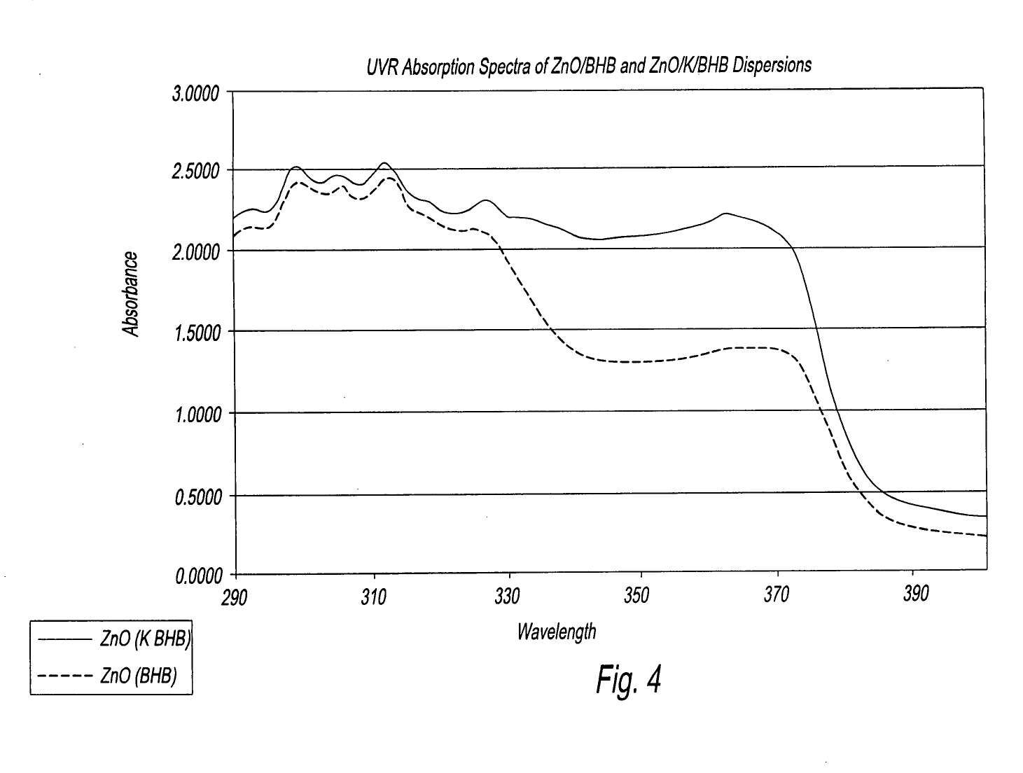

Examples of SPF scans generated by the Labsphere 1000S UV Transmittance Analyzer are shown in FIGS. 2-4. SPF corresponds to absorbance in the UVB region, i.e. approximately 290 nm-320 nm. As shown in FIGS. 2-4, the dispersions of the present disclosure, with both the semiconductor and phenyl compound, exhibit significantly enhanced UVB absorption characteristics over dispersions without the phenyl compounds.

One possible method to confirm the in vitro SPF results was to compare the dielectric constant of the various dispersions. Solvent polarity can affect the UV absorption spectrum of sunscreen active materials, in that generally increasing polarity enhances sunscreen performance. Therefore, knowledge of solvent polarity, expressed as the dielectric constant, helps to understand simple systems such as the dispersions listed above. It is important to note that factors other than particle size affect the dielectric constant of the ZnO powders. These factors include various crystal lattice defects and unintentional doping. The polarity of several carrier oils of the present disclosure are shown below in Table IV.

TABLE IV
Solvent Polarity

| Solvent | Dielectric Constant |
|---|---|
| Isopropyl Myristate | 3.25 |
| Isononyl Isonanoate | 3.25 |
| Ethylhexyl Benzoate | 4.61 |
| Butyloctyl Salicylate | 5.27 |
| Ethylhexyl Salicylate | 6.25 |

Determining the polarity of a mixture or an emulsion can be performed in various ways. For example, determining a polarity can include measuring a property that is a function of polarity, such as a dielectric constant. Measurement of a dielectric constant of a liquid can be performed by various sensors, such as immersion probes, flow-through probes, and cup-type probes, attached to various meters, such as those available from the Brookhaven Instruments Corporation of Holtsville, N.Y. (e.g., model BI-870) and the Scientifica Company of Princeton, N.J. (e.g. models 850 and 870). For consistency of comparison, preferably all measurements for a particular filter system are performed at substantially the same sample temperature, e.g., by use of a water bath. Generally, the measured dielectric constant of a substance will increase at lower temperatures and decrease at higher temperatures.

Data in Table V shows that the trend in polarity of the dispersions of the present disclosure as measured by dielectric constant matches the trends observed for SPF, including the small decrease when Kraton is added to the polar solvent. It is remarkable that the addition of ZnO powder with its low dielectric constant of 3.83 should boost the overall dielectric constant of the dispersions. For the dielectric constants of the ZnO/Kraton/solvent dispersions to be of such high magnitude, there may be a change in dispersion polarity at the molecular (crystalline lattice) level.

TABLE V
Polarity of Dispersions Measured by Dielectric Constant

Dielectric Constant

| Sample | BHB | OS |
|---|---|---|
| Neat | 5.27 | 6.25 |
| +Kraton | 5.21 | 5.98 |
| +Kraton + ZnO | 8.30 | 10.63 |

ZnO Powder (Undried) Dielectric Constant: 3.83

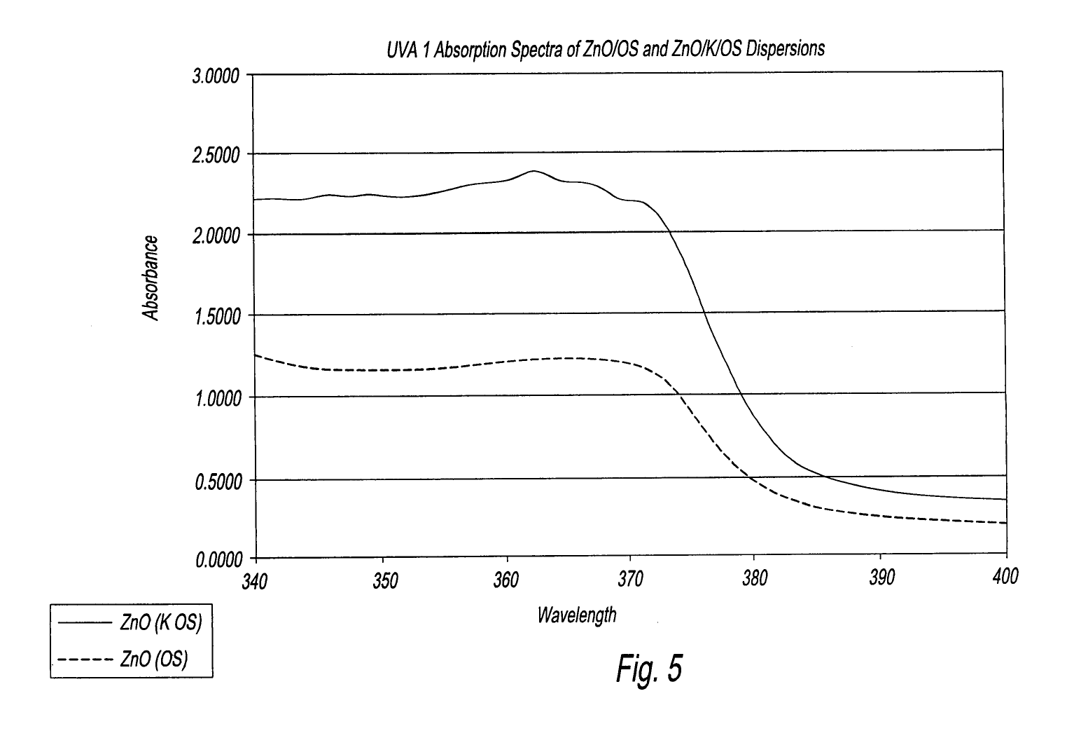

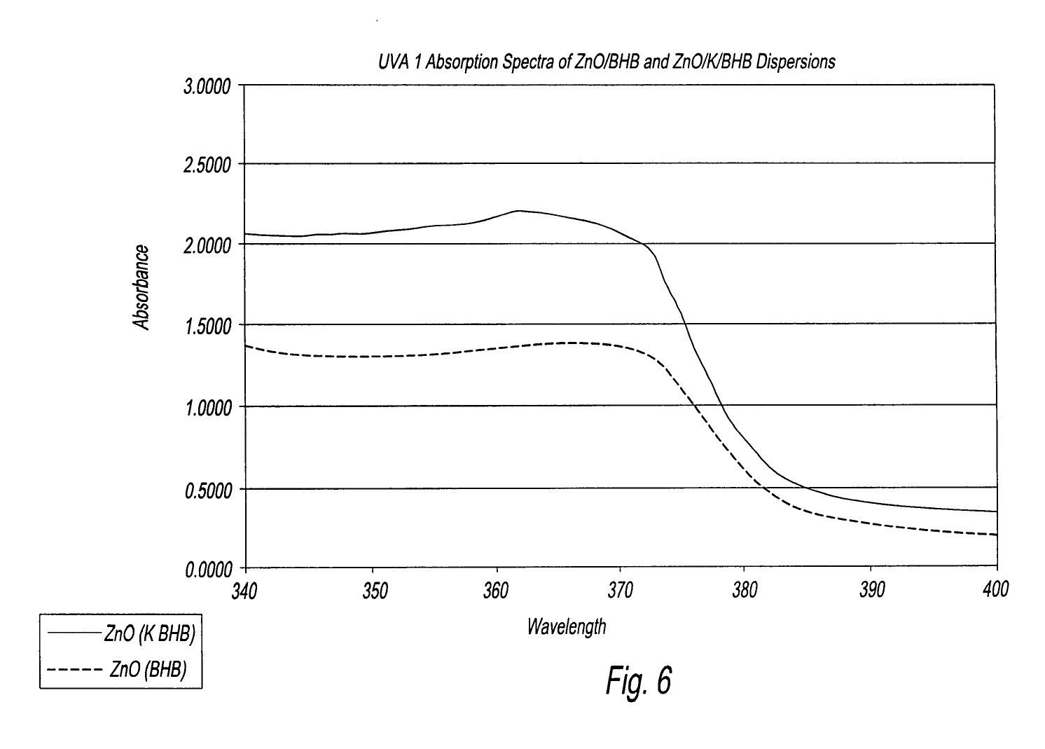

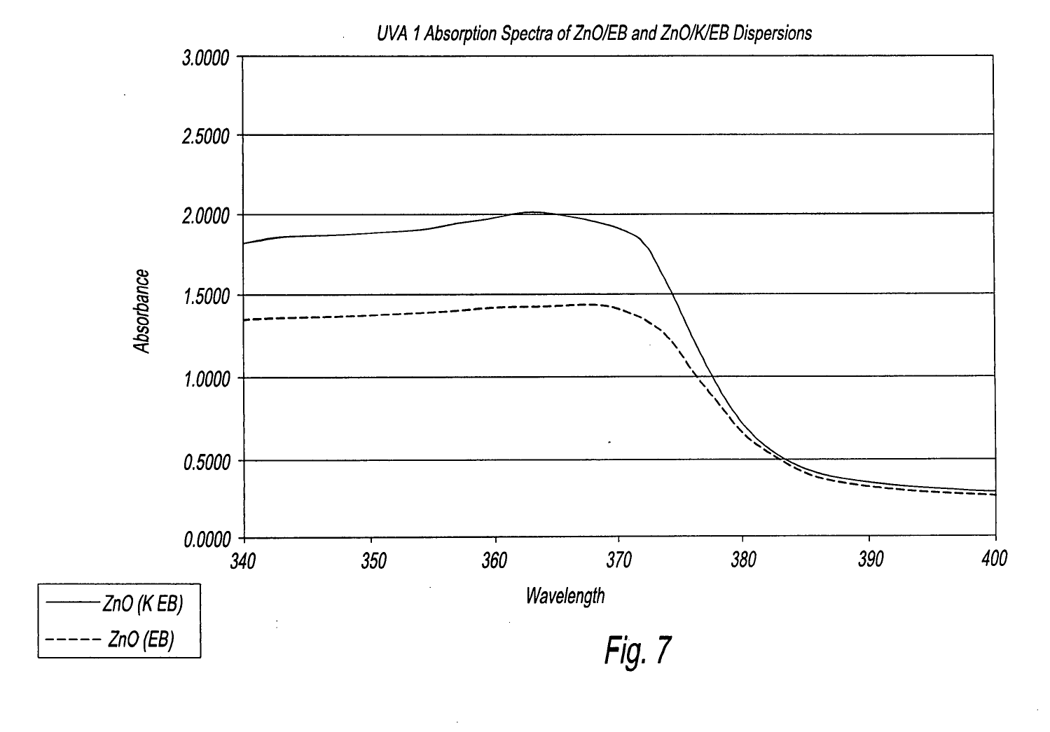

As previously discussed, the dispersions of the present disclosure surprisingly exhibited a significant increase in UVA absorption between 320 nm and 400 nm when comparing dispersions with and without phenyl compounds, as shown in FIGS. 5-7. Obviously, this can be a highly beneficial property of sunscreen compositions. Most notable was the increase in absorption for the UVA1 region of 340 nm-400 nm. A peak appears in the spectra with an approximate initial rise beginning at 360 nm, reaches a maximum that depends on presence of Kraton, and then declines sharply at about 370 nm, and then tails out through 400 nm. As shown in Table VI, the magnitude of the difference in absorption was surprising, with values increasing from 38-93% as solvent polarity increases. Photoactivity was definitely enhanced in the overall UVA region, and especially in the UVA1 region.

TABLE VI
Effect of Kraton on Maximum UVA1 Absorbance of ZnO/Solvent

| | EB | | BHB | | OS | |
|---|---|---|---|---|---|---|
| Sample | λnm | Abs. Max | λnm | Abs. Max | λnm | Abs Max |
| ZnO | 366 | 1.46 | 365 | 1.56 | 365 | 1.23 |
| ZnO + | 362 | 2.02 | 362 | 2.20 | 362 | 2.38 |
| Kraton | | | | | | |
| Difference | | 38.4% | | 41.0% | | 93.5% |

In addition to magnitude of absorption in the UVA region, there was a notable and surprising increase in the breadth of the absorption band. To characterize the expansion towards 400 nm, a fixed absorbance value of 0.5 units was selected and the corresponding wavelength was recorded as shown in Table VII. The data indicated that the influence of Kraton on ZnO caused an increase in absorbance wavelength thereby expanding the range of absorption efficacy of ZnO in the dispersions. In summary, absorption results in terms of magnitude and breadth in the UVA and UVA1 absorption region support a synergistic enhancement to ZnO photoactivity.

TABLE VII
Effect of Kraton on Breadth of UVA1
Absorbance of ZnO/Solvent Dispersions

| | λnm for 0.5 | λnm for 0.5 | λnm for 0.5 |
|---|---|---|---|
| Sample | Abs EB | Abs BHB | Abs in OS |
| ZnO | 383.5 | 382.5 | 379.5 |
| ZnO + Kraton | 384.0 | 384.5 | 385.0 |
| Difference | +0.5 | +2.0 | +5.5 |

To further characterize the photoactivity of the ZnO dispersions, a microwave oven was used as a low energy excitation source. Dielectric heating (also known as electronic heating, RF heating, high-frequency heating) is the phenomenon in which radiowave or microwave electromagnetic radiation heats a dielectric material, especially as caused by dipole rotation. The frequencies used in microwave dielectric heating are 918 MHz and 2450 MHz. Domestic microwave ovens employ 2450 MHz. A Panasonic Microwave Oven 1100 Watt High Power was utilized for these studies. Microwave irradiation induces charged particles to migrate or rotate, which results in polarization of polar particles, and the lag between this polarization and rapid reversals of the microwave field creates friction among molecules to generate heat. In the dispersion systems, the electrons in ZnO and Kraton may vibrate intensely upon absorption of microwaves, and the electrons in the polar solvent may vibrate and rotate intensely, thus generating heat of friction.

The amount of microwave energy absorbed by a given specimen (or “load”) depends on many factors. Among these are the size of the load, its orientation with respect to the waves, and the dielectric and thermal properties of the material. Depending upon the material, microwaves may be reflected, passed through, or absorbed. The absorbed microwave energy causes dipolar molecules to rotate or vibrate at the rate of 2.45 billion cycles per second. The interaction between the rotating dipolar molecules, ions and non-moving molecules induces friction, which in turn produces the heat that warms the dispersion.

Commercially available ZnO for personal care use has crystal lattice type defects that vary significantly from manufacturer to manufacturer. It is known that ZnO powder alone is transparent to microwave energy for electronic transitions to excited states in the conduction band. However, it is not transparent to vibrational modes of excitation which occur at lower valence band energy levels, and it is not transparent to the magnetic portion of the electromagnetic field.

Experimental conditions for the microwave studies were conducted as routinely as possible to minimize variations among the data sets. Samples were exposed to 30 seconds of microwave energy and temperature immediately recorded with a Type K thermometer. The maximum temperature value was recorded, and the experiment repeated on n=5 new samples for each data set. In these experiments, we decided not to dry the ZnO powder and use it as is because that is the use mode in manufacturing for formulated product. IR results confirmed the presence of water molecules in the ZnO powder. Thermogravimetric analysis indicated 0.37 wt % of water at 100° C., and 0.45 wt % of water at 200° C. The data summarized in Table VIII indicates that the combination of ZnO/Kraton/Polar Solvent was surprisingly much more effective in absorbing microwave energy than either component alone. The trend in microwave energy absorption among the dispersions followed the trends noted for SPF, UVA, UVA1, and dielectric constant.

TABLE VIII
Absorption of Microwave Energy bv ZnO Dispersions

Maximum Temperature (° C.)

| Sample | EB | BHB | OS |
|---|---|---|---|
| Solvent Neat | 55.2 ± 1.6 | 65.62 ± 1.8 | 74.8 ± 1.9 |
| | std. dev. | std. dev. | std. dev. |
| Solvent + ZnO + | 106.4 ± 2.7 | 110.02 ± 3.1 | 141.4 ± 2.7 |
| Kraton | std. dev. | std. dev. | std. dev. |

Kraton Powder: 28.4° C. ± 1.5 std. dev.
ZnO Powder (undried): 43.2° C. ± 0.8 std. dev.

The next step involved adding the ZnO/Kraton/BHB and ZnO/Kraton/OS blends to sunscreen formulations for in-vitro and in-vivo testing to help achieve maximum SPF at very water resistant conditions. Although in-vitro SPF was determined using the Labsphere 1000S gave high (unrealistic) values for SPF, it was useful as a relative gauge for formulation development. Formulations were sent to an independent testing facility for in-vivo very water resistant testing according to the method outlined in the Food and Drug Administration (FDA) Monograph for sunscreen testing published in the Federal Register, Vol. 64, No. 98, May 21, 1999, which is incorporated by reference herein.

Prior to this work, several non-ZnO formulations were sent to an independent laboratory for in-vivo SPF very water resistant (VWR) testing. The non-zinc sunscreen formulations were oil-in-water emulsions which included the normally expected additives of emulsifiers, thickeners, stabilizers, film formers, and skin conditioning agents. No formulation passed the SPF VWR test. In several ZnO studies, the additional sunscreen active agents in the formulations included homosalate (10-12%), octisalate (5%), oxybenzone (6%), avobenzone (3%), and octocrylene (6-10%). The in-vivo SPF test results from an independent laboratory were quite surprising in that lower amounts of organic sunscreen agents were used in conjunction with the ZnO dispersions to achieve significantly higher SPF and PFA results as shown in Table IX.

TABLE IX
Evaluation of Sunscreen Lotion Containing ZnO/K/Polar Solvent Dispersions

| | | | | |
|---|---|---|---|---|
| Sunscreen Actives | Avobenzone | 3.0% | Octisalate | 5.0% |
| | Homosalate | 10.0% | Oxybenzone | 6.0% |
| | Octocrylene | 6.0% | Zinc Oxide | 3.0% |

| In Vivo Test | Method | Result |
|---|---|---|
| SPF Static | FDA Monograph, 3 Subjects, UVB Efficacy | 115 ± 5 SPF units |
| SPF VWR | FDA Monograph, 20 Subjects, UVB Efficacy | 102.63 average |
| | | 100.33 @ 95% C.L. |
| PFA | JCIA Method, 10 Subjects, UVA Efficacy | 45.58 average |

Another indicator of performance is sunscreen efficiency, which is a ratio of SPF units to amount of sunscreen active. Commercially available product with somewhat similar levels of organic sunscreen actives and no metal oxide sunscreen actives generally have an SPF VWR rating of 80-85, yielding a sunscreen efficiency of 2.4-2.5:1. These non-zinc products contain homosalate levels at 12-15%. Sunscreen efficiency in the sunscreen compositions of the present disclosure, which contain a combination of organic UV filters and unique zinc oxide dispersions, was an impressive 3.0:1. The SPF VWR 100 sunscreen formulation containing ZnO is the only combination product with organic sunscreen filters and metal oxide achieving remarkable and surprisingly high SPF at water resistant test conditions and broadest spectrum UVR absorption. This sunscreen efficiency ratio demonstrated the “end product” performance value and of having enhanced photoactivity of semiconductors.

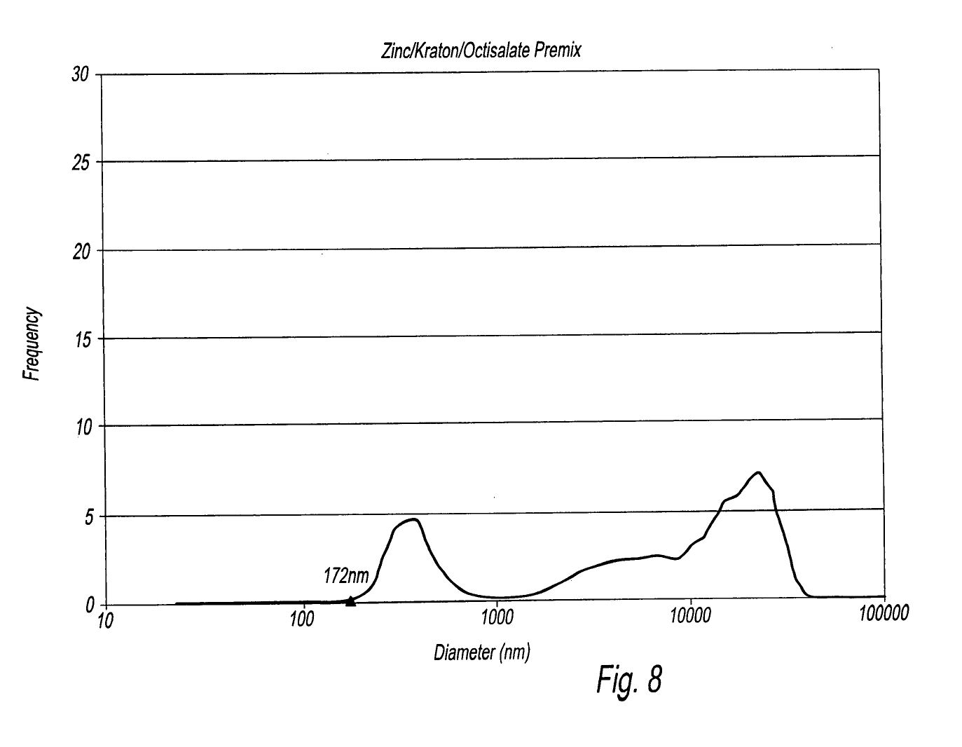

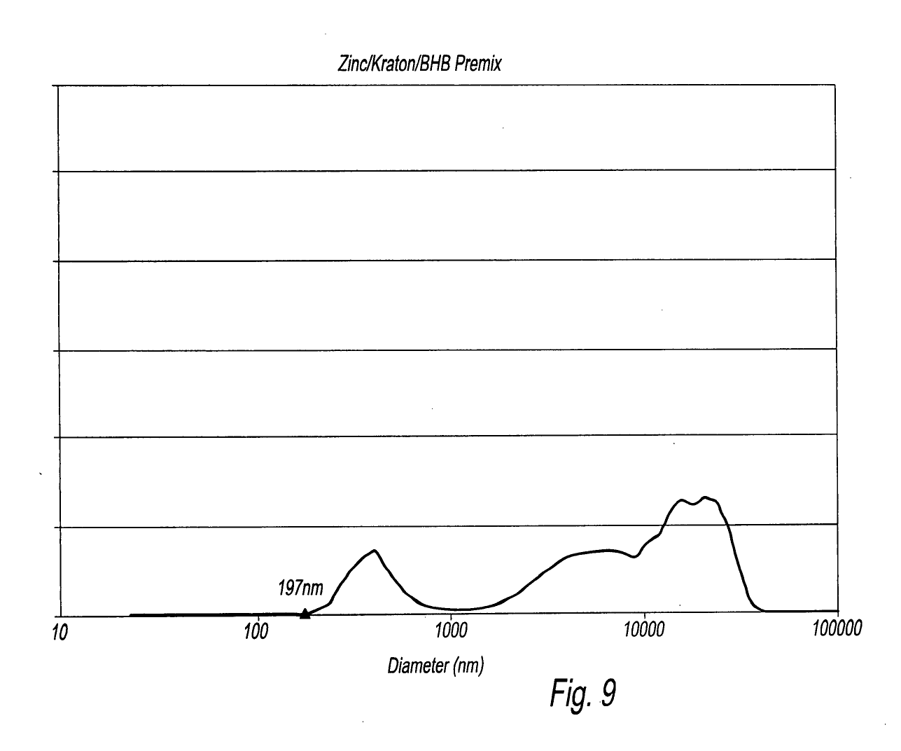

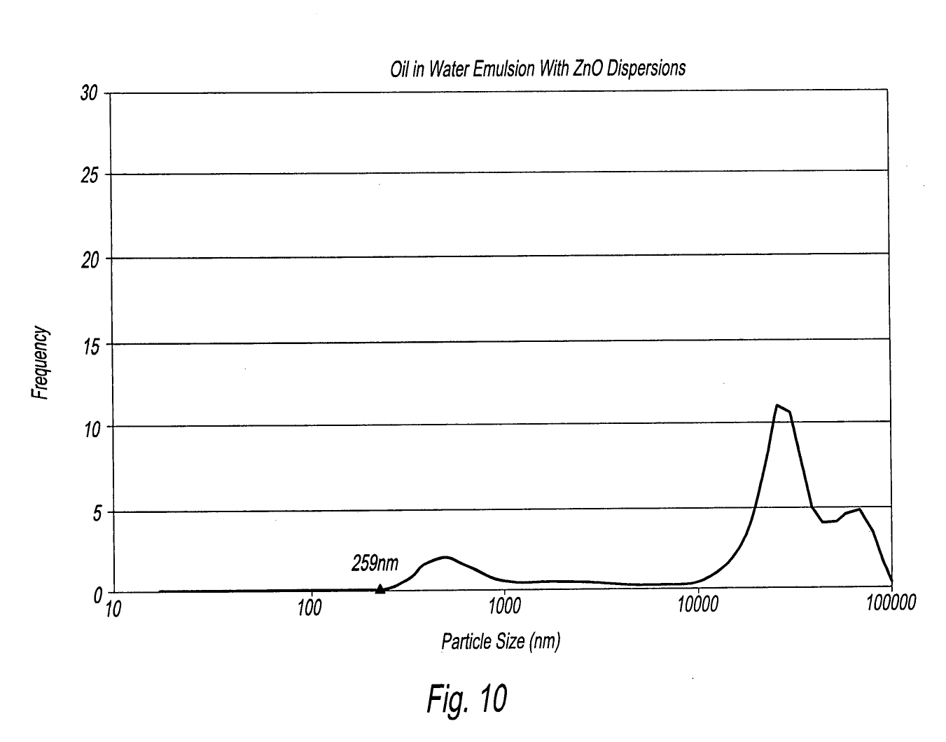

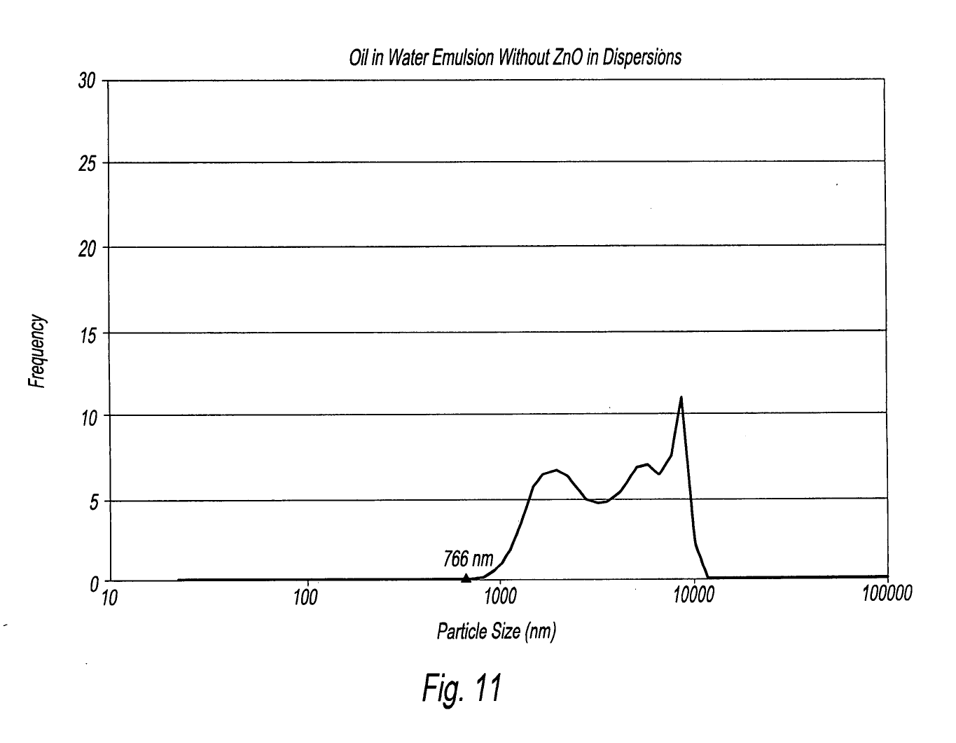

One other advantage of the zinc oxide dispersions is that the system allows the zinc oxide crystals to remain as aggregates with particle sizes greater than 100 microns. This is significant because of concerns raised about nano-sized particles. Results from particle size analyses using a Horiba LA-920 indicated that there are no nanoparticles present in the dispersions or finished formulation. A comparison of FIGS. 8-11 indicated the presence of a peak associated with ZnO when comparing samples of ZnO to the control sample which is the oil-in-water emulsion without ZnO present. The very first particle size recorded for each sample is presented in Table X.

TABLE X
Smallest Particle Size Detected

| Sample | Particle Size (nm) |
|---|---|
| ZnO/Kraton/BHB Dispersion | 197 |
| ZnO/Kraton/OS Dispersion | 172 |
| O/W Emulsion with ZnO Dispersions | 259 |
| O/W Emulsion without ZnO, with | 766 |
| Kraton/Solvents | |

The present invention having been thus described with particular reference to the preferred forms thereof, it will be obvious that various changes and modifications may be made therein without departing from the spirit and scope of the present invention as defined in the appended claims.

What is claimed is:

1. A dispersion, comprising:
a semiconductor;
a compound having multiple phenyl rings; and
a polar carrier oil.

2. The dispersion of claim 1, wherein said semiconductor is selected from the group consisting of zinc oxide, titanium oxide, and a combination thereof.

3. The dispersion of claim 1, wherein said phenyl compound is selected from the group consisting of benzene sulfonic acids, or salts thereof, styrenic block copolymers with a hydrogenated midblock of styrene-ethylene/butylene-styrene, styrenic block copolymers with a hydrogenated midblock of styrene-ethylene/propylene-styrene, styrene/butadiene/styrene block copolymers, styrene/isoprene/styrene block copolymers, ethylene/butadiene/styrene block copolymers, ethylene/propylene/styrene block copolymer, and any derivatives or combinations thereof.

4. The dispersion of claim 1, wherein said polar carrier oil is at least one ester.

5. The dispersion of claim 1, wherein said semiconductor is present in an amount of about 0.50 wt % to about 50 wt %, based on the total weight of the dispersion.

6. The dispersion of claim 5, wherein said semiconductor is present in an amount of about 20 wt % to about 30 wt %, based on the total weight of the dispersion.

7. The dispersion of claim 1, wherein said phenyl compound is present in an amount of about 0.05 wt % to about 10 wt %, based on the total weight of the dispersion.

8. The dispersion of claim 7, wherein said phenyl compound is present in an amount of about 0.10 wt % to about 5 wt %, based on the total weight of the dispersion.

9. The dispersion of claim 4, wherein said ester is selected from the group consisting of benzoate esters, non-benzoate esters, long-chained benzoate esters, long-chained benzoate esters, long-chained non-benzoate esters, long-chained non-benzoate esters, branched benzoate esters, non-branched benzoate esters, branched non-benzoate esters, non-branched non-benzoate esters, and any combinations thereof.

10. The dispersion of claim 1, wherein said polar carrier oil is present in an amount of about 40 wt % to about 99.50 wt %, based on the total weight of the dispersion.

11. The dispersion of claim 10, wherein said polar carrier oil is present in an amount of about 75 wt % to about 95 wt %, based on the total weight of the dispersion.

12. A sunscreen composition, comprising:
a dispersion, wherein said dispersion comprises a semiconductor sunscreen active, a compound having multiple phenyl rings, and a polar carrier oil; and
an additional sunscreen active.

13. The sunscreen composition of claim 12, wherein said semiconductor sunscreen active is selected from the group consisting of zinc oxide, titanium oxide, and a combination thereof,
wherein said compound having multiple phenyl groups is selected from the group consisting of benzene sulfonic acids, or salts thereof, styrenic block copolymers with a hydrogenated midblock of styrene-ethylene/butylene-styrene, styrenic block copolymers with a hydrogenated midblock of styrene-ethylene/propylene-styrene, styrene/butadiene/styrene block copolymers, styrene/isoprene/styrene block copolymers, ethylene/butadiene/styrene block copolymers, ethylene/propylene/styrene block copolymer, and any derivatives or combinations thereof, and
wherein said polar carrier oil is selected from the group consisting of isopropyl myristate, butyloctyl salicylate, octisalate, isononyl isonanoate, and ethylhexyl benzoate, and any combinations thereof.

14. The sunscreen composition of claim 12, wherein said dispersion is present in an amount of about 1 wt % to about 20 wt %, based on the total weight of the sunscreen composition.

15. The sunscreen composition of claim 12, wherein said additional sunscreen is to selected from the group consisting of homosalate, octisalate, oxybenzone, avobenzone, and octocrylene, and any combinations thereof.

16. The sunscreen composition of claim 12, wherein said additional sunscreen is present in an amount of about 1 wt % to about 35 wt %, based on the total weight of the composition.

17. The sunscreen composition of claim 12, further comprising one or more additives selected from the group consisting of emulsifiers, thickeners, emollients, pH adjusters, stabilizers, and film formers.

18. The dispersion of claim 1, wherein the minimum aggregate particle size of said semiconductor is 100 nanometers.

19. The sunscreen composition of claim 12, wherein the minimum aggregate particle size of said semiconductor is 100 nanometers.

20. A sunscreen composition, comprising:
a dispersion, wherein said dispersion comprises a semiconductor sunscreen active selected from the group consisting of zinc oxide, titanium oxide, and a combination thereof, a compound having multiple phenyl rings selected from the group consisting of sodium polystyrene benzene sulfonate, ethylene/butadiene/styrene block copolymer, and a combination thereof, and a polar carrier oil selected from the group consisting of isopropyl myristate, butyloctyl salicylate, octisalate, isononyl isonanoate, and ethylhexyl benzoate, and any combinations thereof; and
an additional sunscreen active.
